(19) United States
(12) Patent Application Publication
(10) Pub. No.: US 2025/0025086 A1
(43) Pub. Date: Jan. 23, 2025

(54) MULTI-CONTACT INTRACEREBRAL FUNCTIONAL EXPLORATION PROBE

(71) Applicant: NEXT NEUROTECH, Nîmes (FR)

(72) Inventors: Yoann VALORGE, RANCHOT (FR); Christophe MOUREAUX, BESANÇON (FR); Sébastien DAVID, BESANÇON (FR)

(21) Appl. No.: 18/274,159

(22) PCT Filed: Jan. 20, 2022
(86) PCT No.: PCT/EP2022/051234

§ 371 Date: Jul. 25, 2023

(30) Foreign Application Priority Data
Jan. 27, 2021 (FR) FR2100760

(51) Int. Cl. A61B 5/293

(52) U.S. Cl. CPC A61B 5/293

(57) ABSTRACT

Disclosed is a multi-contact probe (100) for intracerebral functional exploration, comprising: a cylindrical distal portion (101) comprising at least one intracerebral contact (11), a cylindrical proximal portion (103) comprising at least one connector contact (77), and a non-cylindrical connection portion (102) connecting the distal portion (101) and the proximal portion (103), the distal portion (101), the proximal portion (103) and the connection portion (102) being formed by a multi-layer film (5) comprising a substrate (20) and at least one conductive layer (10) deposited on the substrate (20), the substrate (20) being produced in at least one polymer material, the at least one conductive layer (10) comprising at least one transmission track (12) and, in the distal portion, the at least one intracerebral contact (11) as well as, in the proximal portion, the at least one connector contact (77).

### TECHNICAL FIELD

The present invention concerns the medical field. In particular, the invention concerns a multi-contact intracerebral functional investigation probe and a method of manufacturing such a probe. The invention also concerns a device for intracerebral functional investigation and/or stimulation and/or treatment by radiofrequencies including such a probe.

### PRIOR ART

In order to diagnose or to treat certain pathologies, such as drug-resistant epilepsy or Parkinson's disease, for example, it is useful to employ intracerebral probes implanted, generally temporarily, in the organism of a patient.

Such probes may be implanted in the brain of a patient to record the intracerebral electrical activities during stereo-encephalography (SEEG) or to produce electrical stimulation and, where appropriate, to identify an anomaly and then possibly to treat it.

Intracerebral probes are implanted in accordance with an implantation scheme worked out previously for each patient as a function of hypotheses as to the origin of the pathology.

The intracerebral probes are generally devices from 15 to 100 cm long and, from the point of view of the surgeon, divided into three parts:
the “distal” part, that is to say the part at a distance from the surgeon, intended to be implanted in the organism of the patient,
the “proximal” part, that is to say the part near the surgeon, intended to connect the probe to a recording or processing device and/or a signal transmission device, and
an intermediate part connecting the distal part and the proximal part, this intermediate part generally being cylindrical.

The distal part consists of alternating conductive zones connected by conductive tracks to a connector in the proximal part.

To manufacture the distal part of the probes it is known to use a cylindrical thermoplastic polymer material tube on which metal rings are mounted.

Another known method of manufacture consists in juxtaposing and welding or gluing together a succession of cylindrical parts respectively including a thermoplastic polymer material and a metal.

In both cases the number of conductive zones is limited by the robustness of the probe assembly. In fact, in order to guarantee that these probes are safe from the sanitary point of view they must be sufficiently robust to be able to be withdrawn without leaving foreign bodies in the brain of the patient. The number of conductive zones is therefore limited by the robustness of the probe assembly.

Thus there exists a need for a multi-contact intracerebral functional investigation zone that is robust and reliable and that is able to include a large number of conductive zones.

### STATEMENT OF INVENTION

The present invention addresses this requirement in whole or in part thanks to, in one of its aspects, a multi-contact probe for intracerebral functional investigation and/or stimulation and/or treatment by radiofrequencies, including:
a distal part of cylindrical shape including at least one intracerebral contact intended to be implanted in the brain of a patient,
a proximal part of cylindrical shape including at least one connector contact intended to be connected to at least one device for recording and/or stimulation and/or treatment outside the body of the patient, and
a connecting part of non-cylindrical, in particular flat shape connecting the distal part and the proximal part.

The distal part, the proximal part and the connecting part comprise a multilayer film including a substrate and at least one conductive layer deposited on the substrate. The substrate includes at least one polymer material and is preferably made in one piece. Said at least one conductive layer includes at least one transmission track and, in said distal part, said at least one intracerebral contact as well as, in the proximal part, said at least one connector contact, each transmission track being connected to an intracerebral contact in the distal part and to a connector contact in the proximal part.

When the substrate is manufactured in one piece the distal part, the proximal part and the connecting part are therefore obtained from the same substrate.

Said at least one intracerebral contact is intended to be in contact with the brain of a patient in order to pick up an electrical signal characteristic of a cerebral activity or to send an electrical signal into a zone of the brain of the patient to perform a treatment.

The substantially flat shape of said connecting part confers on it high flexibility, which facilitates its manipulation by medical personnel and ensures comfort for the patient, in particular when the probe is implanted in the brain of a patient for several days.

By “cylinder” is meant a surface with parallel straight line generatrices, that is to say a surface constituted of parallel lines in space. The cylinder has two openings at the ends, each opening being adapted to be inscribed in a plane parallel to the other one. The section of the cylinder may take various shapes, such as for example circular, substantially circular, ovoid, oval, square, rectangular, star-shaped or other shape. The section of the cylinders forming the distal part and the proximal part is preferably substantially circular.

By “at least one conductive layer” is meant at least one layer of material with good electrical conductivity, for example a layer of metal, graphite or any other material with good electrical conductivity, in particular a biocompatible material in the zones liable to come into contact with the patient. Said at least one conductive layer may form only lines forming the transmission tracks and, in particular at the ends of the latter, the intracerebral and connector contacts.

The or each conductive layer may include gold and/or platinum and/or copper and/or iridium and/or any other biocompatible conductive material, preferably platinum and/or gold.

The or each conductive layer preferably has a thickness between approximately 1 μm and 1000 μm inclusive, preferably between approximately 5 μm and 15 μm inclusive.

The multi-contact intracerebral functional investigation probe according to the invention is reliable. In fact, given that the distal part of the probe includes a limited number of parts fixed together, in particular a single part, there is a low risk of the probe coming apart is during its insertion or extraction.

The fact that the or each transmission track is connected to an intracerebral contact and to a connector contact enables transmission of the signal or signals picked up by said intracerebral contact. When there is a plurality of intracerebral contacts each of them is preferably connected to a distinct transmission track that is specific to it, that is associated with it, each of them being also preferably connected to a distinct connector contact that is specific to it, that is associated with it.

The probe may include between 1 and 60 intracerebral contacts, in particular between 2 and 20 intracerebral contacts.

In one embodiment each intracerebral contact extends over the whole of the circumference of the distal part of the probe.

Alternatively, at least one intracerebral contact extends over only part of the circumference of the distal part of the probe.

When the probe includes a plurality of intracerebral contacts they may be at a distance from one another, intracerebral contacts that are adjacent two by two being separated by an inter-contact distance that may be constant or vary. An insulative layer may be present in the space between two adjacent intracerebral contacts.

The intracerebral contacts deposited may have identical or different lengths. By “length” is meant the length of said at least one intracerebral contact in a direction parallel to a longitudinal axis of the substrate.

Said at least one intracerebral contact may include at least two separate parts.

The intracerebral contact or contacts may have a circular shape with identical or different radii.

The distal part may include a proximal end that is closed, for example by a plug or by the multilayer film itself.

Each intracerebral contact may be connected to a single connector contact by a single transmission track. The connector contact enables connection with the recording and/or stimulation and/or treatment device outside the body of the patient for transmission of the electrical signals in one direction and the other.

Said proximal part and/or said distal part may extend along a longitudinal axis.

A longitudinal axis of this kind may be straight or curved or may include straight portions and curved portions. The longitudinal axis of the proximal and/or distal parts may be that of the substrate, in particular at the time of forming the cylinder forming the proximal and/or distal part, but the orientation of the distal and proximal parts may of course vary, in particular during use of the probe, preferably being mobile relative to one another, in particular with the aid of the connecting part.

It is preferable to insulate from the brain of the patient said at least one transmission track. The multilayer film may include, deposited on the substrate, at least one insulative layer of polymer material, preferably a liquid crystal polymer material. In this case said at least one insulative layer may cover at least partly said at least one transmission track.

Said at least one insulative layer advantageously covers the whole of said at least one transmission track except for the ends of said at least one transmission track in contact with said at least one intracerebral contact and with said at least one connector contact. In this way said at least one intracerebral contact and said at least one transmission track are connected by a via, or well, in said at least one insulative layer, said via preferably extending transversely, in particular orthogonally, to said at least one insulative layer. Similarly, said at least one connector contact and said at least one transmission track are connected by a via, or well, in said at least one insulative layer, said via preferably extending transversely, in particular orthogonally, to said at least one insulative layer. On the other hand, said at least one insulative layer is preferably such as not to cover said at least one intracerebral contact so that the latter is in contact with the brain and is able to pick up or transmit electrical signals.

Said at least one insulative layer may include a polymer material selected in the group consisting of liquid crystal polymers, polyamides, silicones and any other biocompatible thermoplastic polymer material, preferably a liquid crystal polymer material.

The insulative layer or layers are for example made of the same material as the substrate.

The insulative layer or layers may be deposited on and then fixed to the substrate by compression and/or heating of the insulative layer or layers.

If necessary, the or each insulative layer preferably has a thickness between approximately 1 μm and 1600 μm inclusive, preferably between approximately 20 μm and 30 μm inclusive.

The substrate preferably includes at least one liquid crystal polymer (LCP) material.

A substrate including at least one liquid crystal polymer material enables good long-term reliability to be conferred on the probe. For example, this enables insertion of the probe in the brain of a patient for several days, or even several months, with high quality electrical signal transmission throughout that period.

The substrate preferably has a thickness between approximately 1 μm and 160 μm inclusive, preferably between approximately 25 μm and 90 μm inclusive.

The distal part has a cylindrical shape, thus that of a cylinder defining an interior cavity. The interior cavity formed by the cylinder of the distal part may be at least partly filled with at least one glue or a polymer material or a composite material, in particular a silicone charged with metal particles. The filling may be homogeneous or heterogeneous.

The probe may include a distal stud. In this case the distal part may have a distal end closed by the distal stud. A distal stud of this kind may be made of at least one conductive material, in particular at least one metal. In this case the distal stud, when conductive, is able to form an intracerebral contact independent of the other intracerebral contact or contacts. In this case, the probe advantageously includes a conductive transmission track in contact with the distal stud, when conductive. The probe may alternatively include a transmission wire connected to the distal stud, for example connected to a connector contact on the proximal part.

When the interior cavity formed by the cylinder of the distal part is at least partly filled with silicone charged with metal particles and the probe includes a conductive distal stud, the silicone charged with metal particles can make it possible to establish an electrical connection between the conductive distal stud and a conductive transmission wire or a conductive transmission track in contact with the charged silicone.

The distal part may include at least one temperature sensor, formed in particular by said at least one conducting layer. In this case the multi-contact intracerebral functional investigation probe enables thermal data on the brain of the patient to be obtained. The or each temperature sensor may be a resistance temperature sensor, for example a platinum resistance sensor. The temperature sensor may include a thermocouple.

In accordance with another of its aspects the invention further has for object, independently of and/or in combination with the foregoing, a multi-contact probe for intracerebral functional investigation and/or stimulation and/or treatment, in particular by radiofrequencies, including a distal part intended to be implanted in the brain of a patient including a distal part of cylindrical shape including at least one intracerebral contact and at least one temperature sensor intended to be implanted in the brain of a patient.

The distal part comprises a multilayer film including a substrate and at least one conductive layer deposited on the substrate. The substrate includes at least one polymer material, preferably in one piece. Said at least one conductive layer includes at least one transmission track, said at least one intracerebral contact and said at least one temperature sensor, each transmission track being connected to an intracerebral contact.

In accordance with another of its aspects the invention further has for object, independently of and/or in combination with the foregoing, a multi-contact probe for intracerebral functional investigation and/or stimulation and/or treatment, in particular by radiofrequencies, including a distal part intended to be implanted in the brain of a patient including a distal part of cylindrical shape including at least one intracerebral contact.

The distal part comprises a multilayer film including a substrate and at least one conductive layer deposited on the substrate. The substrate includes at least one polymer material, preferably in one piece. Said at least one conductive layer includes at least one transmission track and said at least one intracerebral contact, each transmission track being connected to an intracerebral contact.

In accordance with this aspect its distal part has a distal end closed by a distal stud.

In accordance with another of its aspects, the invention further has for object, independently of and/or in combination with the foregoing, a multi-contact probe for intracerebral functional investigation and/or stimulation and/or treatment, in particular by radiofrequencies, including a distal part intended to be implanted in the brain of a patient, of cylindrical shape and including at least one intracerebral contact.

The distal part comprises a multilayer film including a substrate and at least one conductive layer deposited on the substrate. The substrate includes at least one polymer material, preferably in one piece. Said at least one conductive layer includes at least one transmission track and said at least one intracerebral contact, each transmission track being connected to an intracerebral contact.

In accordance with this aspect of the invention the multilayer film has two lateral edges in the distal part, the two lateral edges being in edge to edge contact.

### Method of Manufacturing a Multi-Contact Intracerebral Functional Investigation Probe

In accordance with another of its aspects, in combination with the foregoing, the invention further has for object a method of manufacturing a multi-contact probe for intracerebral functional investigation and/or stimulation and/or treatment, in particular by radiofrequencies, as described above, the method including the following steps:
a) Step a: forming a multilayer film by depositing flat, on at least a part of the substrate, at least one conductive layer forming at least one intracerebral contact, at least one connector contact and at least one transmission track, each transmission track being connected to an intracerebral contact and to a connector contact,
b) Step b: forming a cylinder extending along a longitudinal axis from at least one first part of the multilayer film intended to form said distal part of the probe intended to be implanted in the brain of a patient, in order to obtain the latter part,
c) Step c: forming a cylinder extending along a longitudinal axis from at least one second part of the multilayer film intended to form said proximal part of the probe intended to be connected to at least one device for recording and/or stimulation and/or treatment outside the patient, in order to obtain the latter part.

By “depositing flat” is meant that during step a including depositing the conductive layer or layers the substrate has a substantially flat shape.

Steps b and c may be carried out simultaneously.

A single substrate, preferably made in one piece, is used to form said distal part, said connecting part and said proximal part. The method therefore enables manufacture of a multi-contact intracerebral functional investigation probe with a limited number of components fixed together, in particular a single component, which limits the risks of disintegration of the latter during insertion or extraction of the distal part of the probe in or from the brain of a patient and limits its overall size.

The distal part, the proximal part and the connecting part therefore preferably constitute a single part.

The distal and proximal parts are advantageously identical apart from the geometry of the intracerebral contacts and the connector contacts.

The substrate is preferably made from at least one liquid crystal polymer (LCP) material. In fact, liquid crystal polymer materials are neither damaged nor dissolved by organic solvents used in micro-manufacturing, such as for example an alcohol, an acetone, a photo-sensitive resin, a photo-sensitive resin revealer/dissolver or an acid etching agent for metals. This resistance to solvents enables said at least one conductive layer to be deposited with great precision, for example with the aid of a centrifugal coating process, a metallisation process, a photolithography process or a dry or wet etching process.

Using a substrate made of at least one liquid crystal polymer material therefore facilitates and improves the deposition of said at least one conductive layer.

Step a may consist in forming one or more layers, in particular one or more conductive layers, on the substrate. To this end step a may consist in producing a stack (or a superposition) of different layers, in particular different conductive layers.

Said at least one connector contact is preferably formed on said second part.

Step a may include, after depositing said at least one conductive layer on the substrate, compression and/or heating of said at least one conductive layer on the substrate in such a manner as to fix said at least one conductive layer on the substrate.

Said at least one conductive layer is preferably produced separately and then deposited on the substrate.

The method may include, in particular before step b, a step of finishing said at least one conductive layer, such as for example stripping and/or wet or dry etching.

It is preferable or even necessary to insulate said at least one transmission track from the brain of the patient. The method may therefore include the step consisting in depositing at least one insulative layer of a polymer material on the substrate before step b, said at least one insulative layer covering at least partly said at least one transmission track.

In this way each signal picked up by the or each intracerebral contact can be transmitted via the transmission track that is associated with it without being degraded or disturbed by any electrical contact between the transmission track and the brain of the patient.

The flat substrate is preferably elongate along a longitudinal axis.

Step a may be performed in such a manner that said at least one intracerebral contact has a surface with a transverse width between approximately 0.1 mm and 10 mm inclusive, preferably equal to approximately 2 mm, and a longitudinal length between approximately 0.1 mm and 10 mm inclusive, preferably equal to approximately 2 mm.

By “transverse width” is meant the width of said at least one intracerebral contact in a direction transverse to the longitudinal axis of the substrate.

By “longitudinal length” is meant the length of said at least one intracerebral contact in a direction in parallel to the longitudinal axis of the substrate.

Said at least one intracerebral contact may extend over the whole or only part of the transverse width of the substrate.

When a plurality of intracerebral contacts are deposited on the substrate they may be deposited at a distance from one another, adjacent intracerebral contacts being separated by an inter-contact distance that may be constant or vary. It is possible to deposit an insulative layer at the location of the space between two intracerebral contacts.

Said at least one intracerebral contact may include at least two separate parts and extend from a lateral end of the substrate.

The intracerebral contacts may be deposited in such a manner as to have a circular shape with identical or different radii.

The method may include before the shaping step b a step of cutting out the substrate. This cutting out may be performed before or after step a.

In one particular embodiment, during step a at least one temperature sensor is deposited on the substrate by gluing, welding or stacking, preferably on said first part of the film. In this case the multi-contact intracerebral functional investigation probe makes it possible to obtain thermal data on the brain of the patient. The or each temperature sensor may be a resistance temperature sensor, for example using a platinum resistance. A sensor of this kind may be formed by at least one conductive layer deposited during step a. The temperature sensor may include a thermocouple.

Said first part and/or said second part of the film may have two lateral edges. In this case step b may include at least partial rolling of said first part and/or said second part of the film on itself.

In one embodiment said first part and/or said second part of the multilayer film including two lateral edges, step b and/or step c include(s) at least partial rolling of said first part and/or said second part of the multilayer film on itself or themselves in such a manner as at least partly to superpose said lateral edges.

By “two lateral edges” is meant, for each edge, including the lateral end part of the multilayer film and also a part of the surface of the multilayer film near that lateral end.

Said lateral edges may be glued and/or welded together.

In another embodiment, said first part and/or said second part of the film including two lateral edges, step b and/or c includes at least partial rolling of said first part and/or said second part of the film in such a manner as to bring said lateral edges into edge-to-edge contact.

The substrate may include on at least one of said lateral edges at least one window, step a being performable in such a manner as to deposit said at least one conductive layer outside said at least one window. The presence of a window or windows can make it possible to facilitate the fixing together of said edges, the or each window increasing the fixing length between the two lateral edges.

In one or the other of these embodiments said first part and/or said second part of the multilayer film may be rolled by successively inserting said first part and/or said second part of the film into at least one truncated cone, the diameter of the equivalent circular section of said first part and/or said second part of the film being reduced after successive insertion in each truncated cone.

In another embodiment, the substrate having within its thickness an interior cavity closed laterally but open at one longitudinal end at least, step b consists in filling said interior cavity of the substrate with at least one material, in particular at least one biocompatible material.

After step b, including in particular rolling of said first part of the film, the method may include at least partial filling of an interior cavity formed by the cylinder of the distal part. Such filling may be performed by injection of at least one glue or polymer or composite material, in particular silicone charged with metal particles.

When the method includes, after the at least partial rolling of said first part of the film, at least partial filling of an interior cavity formed by the cylinder of the distal part, this filling is performed in particular near said lateral edges, preferably by injection of at least one glue or a polymer material or a composite material, in particular a silicone. This filling may be homogeneous or heterogeneous.

The method advantageously includes, in particular during or after step b, a step consisting in inserting a distal stud, in particular one including at least one conductive material, for example at least one metal, in a distal end of the multilayer film.

A distal stud of this kind may be fixed by gluing, welding, molding, overmolding and/or mechanical fixing, for example by forcible insertion. A distal stud of this kind may longitudinally project from the distal end of the multilayer film and form the distal end of the distal part. When it is made at least partly of metal the distal stud advantageously forms an intracerebral contact independent of said at least one intracerebral contact deposited on the substrate during step a, that is to say the distal stud is not in contact with said at least one intracerebral contact or with said at least one transmission track. In this case step a may include depositing onto the substrate at least one conductive layer forming at least one distal transmission track, said at least one distal transmission track being intended to be in contact with said distal stud after insertion of the latter in said distal end.

Said at least one conductive layer may be deposited during step a to form at least one connector contact, preferably on said second part, connected to said at least one distal transmission track.

A plug may be inserted in a proximal end of the distal part, for example by filling, molding, overmolding and/or by mechanical fixing, in particular by forcible insertion, gluing or welding. The plug need not project beyond the proximal end, in particular longitudinally.

Independently of or in combination with the foregoing, the invention further has for object a method of manufacturing a multi-contact probe for intracerebral functional investigation and/or stimulation and/or treatment, in particular by radiofrequencies, including a distal part intended to be implanted in the brain of a patient, the method including the following steps:
a) Step a: forming a multilayer film by depositing flat, on at least a part of the substrate, at least one conductive layer forming at least one intracerebral contact and at least one transmission track, each transmission track being connected to an intracerebral contact, said first part of the film having two lateral edges,
b) Step b: forming a cylinder extending along a longitudinal axis from said first part of the film in order to obtain the distal part, step b including at least partial rolling of said first part of the film on itself, said first part of the film being rolled by successive insertion of said first part in at least two truncated cones, the diameter of the equivalent circular section of said first part of the film being reduced after that successive insertion in each truncated cone.

The method may include the production of a proximal part of the multi-contact intracerebral functional investigation probe. In this case said multilayer film may include a second part intended to form said proximal part, said at least one conductive layer being adapted to form at least one connector contact on this second part, said at least one connector contact being connected to a transmission track.

The method may include a step consisting in forming a cylinder extending along a longitudinal axis from said second part of the film in order to obtain the proximal part, this step including at least partial rolling of said second part of the film on itself, the rolling of said second part of the film being performed by successively inserting said second part of the film in at least two truncated cones, the diameter of the equivalent circular section of said second part of the film being reduced after successive insertion in each truncated cone.

### Multi-Contact Intracerebral Functional Investigation Device

In accordance with another of its aspects, in combination with the foregoing, the invention further has for object a multi-contact device for intracerebral functional investigation and/or stimulation and/or treatment, in particular by radiofrequencies, including at least one multi-contact probe for intracerebral functional investigation as defined above and at least one device for recording and/or stimulation and/or treatment of the patient connected to said at least one probe.

The device may be connected to a third party data processing device adapted to process signals picked up by the intracerebral contact or contacts and transmitted by the associated transmission track or tracks.

The connector contact or contacts present on the proximal part enable transmission of electrical signals from the probe to the recording and/or treatment and/or stimulation device for the patient and/or to the data processing device, and vice-versa.

Such a device can make it possible to obtain a diagnosis of the cerebral activity of the brain, for example a stereo-encephalography (SEEG) and/or treatment of one or more zones of the brain.

Such a device can enable collection of data on the neural activity of a patient.

The device may enable electrical stimulation of the brain of a patient, in particular in the vicinity of said at least one intracerebral contact. This stimulation may be performed using a periodic electrical signal, for example with a frequency between approximately 1 and 1000 Hz inclusive, in particular using a current between approximately 0.1 and 20 mA inclusive.

The device can enable treatment by thermo-coagulation using a high-frequency electrical signal sent into the brain of a patient, in particular near said at least one intracerebral contact, for example at a frequency between approximately 400 kHz and 600 kHz inclusive, in particular with a power between approximately 0.0001 W and 10 W inclusive.

### Intracerebral Treatment or Intracerebral Functional Investigation Method

In accordance with another of its aspects, in combination with the foregoing, the invention further has for object a method of intracerebral functional investigation or stimulation and/or treatment including the following steps:
Step x: inserting at least one multi-contact probe for intracerebral functional investigation and/or stimulation and/or treatment, in particular by radiofrequencies, as defined above, in the brain of a patient,
Step y: connecting said at least one probe to at least one recording and/or stimulation and/or treatment device,
Step z: measuring nervous cerebral activity with the aid of the intracerebral contact or contacts and/or performing electrical stimulation in the brain of the patient and/or performing electrical treatment in the brain of the patient.

### BRIEF DESCRIPTION OF THE DRAWINGS

The invention may be better understood on reading the following detailed description of non-limiting embodiments thereof and examining the appended drawings, in which:

FIG. 1 is a schematic and partial depiction in perspective of an example of a multi-contact probe according to the invention for intracerebral functional investigation and/or stimulation and/or treatment, in particular using radiofrequencies,

FIG. 2 is a schematic and partial depiction in cross section of the part of the multilayer film forming the distal part of FIG. 1,

FIG. 3 is a schematic depiction in perspective of an example of the distal part of the probe according to the invention,

FIG. 4 is a schematic depiction in perspective of another example of the distal part of the probe according to the invention,

FIG. 5 is a schematic depiction in perspective of another example of the distal part of the probe according to the invention,

FIG. 6 is a schematic depiction in perspective of another example of the distal part of the probe according to the invention,

FIG. 7 is a schematic depiction in perspective of another example of the distal part of the probe according to the invention,

FIG. 8 is a schematic depiction in perspective of the distal end of a distal part of an example of a probe according to the invention,

FIG. 9 is a schematic depiction in perspective of the proximal end of a distal part of an example of a probe according to the invention,

FIG. 10 is a schematic depiction in perspective of an example of the proximal part of the probe according to the invention,

FIG. 11 is a schematic depiction in perspective of another example of the proximal part of the probe according to the invention,

FIG. 12 is a block schematic of an example of a method of manufacturing a probe according to the invention,

FIG. 13 is a schematic depiction as seen from above of an example of a multilayer film intended to form a probe according to the invention,

FIG. 14 is a schematic view from above of an example of part of the multilayer film intended to form the distal part,

FIG. 15 is a view similar to FIG. 14 of another example of part of the multilayer film intended to form the distal part,

FIG. 16 is a view similar to FIG. 14 of another example of part of the multilayer film intended to form the distal part and including two temperature sensors,

FIG. 17 is a schematic depiction as seen from above and in isolation of one of the temperature sensors of the FIG. 16 example,

FIG. 18 represents a schematic depiction of the connection of the temperature sensor from FIG. 16,

FIG. 19 is a schematic depiction in perspective of various substeps of an example of performing step b of the method according to the invention,

FIG. 20 is a schematic depiction of filling the cavity of the cylinder formed by the film after step b of the method according to the invention,

FIG. 21 is a schematic depiction in perspective of an example of part of the multilayer film intended to form the distal part of the probe according to the invention,

FIG. 22 is a schematic depiction in perspective of the distal part formed from the multilayer film part from FIG. 21,

FIG. 23 is a schematic depiction as seen from above of another example of a multilayer film intended to form the probe according to the invention,

FIG. 24 is a schematic view in cross section of a distal part made from the multilayer film from FIG. 23, and

FIG. 25 is a schematic depiction of an investigation device according to the invention including a plurality of probes according to the invention during its use.

### DETAILED DESCRIPTION

In the remainder of the description elements that are identical or have identical functions bear the same reference sign. For conciseness in the present description they are not described with reference to each of the figures, only the differences between the embodiments being described.

For clarity, the real proportions have not always been respected in the figures.

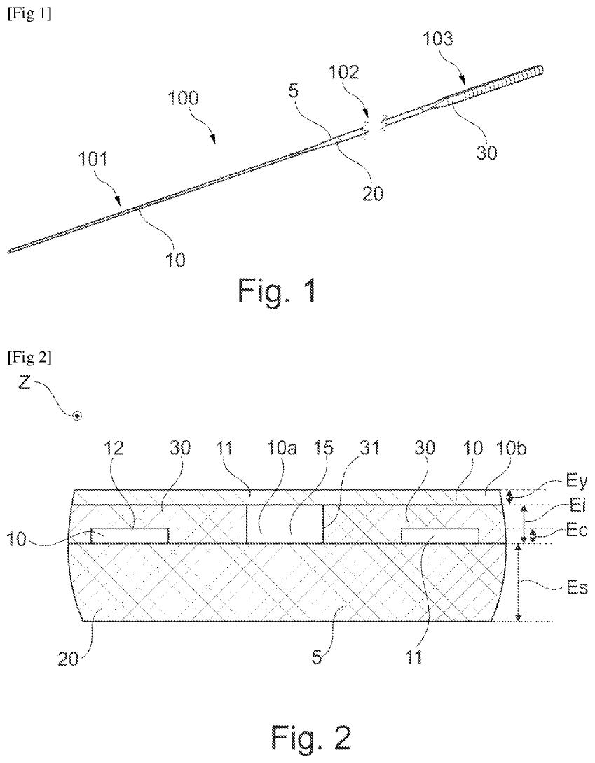

There has been depicted in FIG. 1 an example of a multi-contact probe 100 according to the invention for intracerebral functional investigation and/or stimulation and/or treatment, in particular using radiofrequencies. The probe 100 includes a distal part 101, a proximal part 103 and a connecting part 102 to connect together the distal part 101 and the proximal part 103.

The distal part 101 has a cylindrical shape. It is intended to be implanted in the brain of a patient.

The proximal part 103 has a cylindrical shape and is intended to be connected to at least one recording and/or stimulation and/or treatment device outside the body of the patient, not represented in this figure.

The connecting part 102 has a substantially flat shape.

The distal part 101, the proximal part 103 and the connecting part 102 are formed by a multilayer film 5 including a substrate 20 and at least one conductive layer 10 deposited on the substrate 20.

In this example the substrate 20 is made in one piece and includes at least one liquid crystal polymer material.

In this example the multilayer film 5 also includes an insulative layer 30.

As can be seen in FIG. 2, the conductive layer 10 forms on the distal part 101 a plurality of intracerebral contacts 11 and a plurality of transmission tracks 12. In this example the insulative layer 30 covers the transmission tracks 12 in order for them not to be able to come into contact with the brain of the patient. In FIG. 2, to clarify the drawing, only a part of the transmission tracks 12 is represented. In this example each intracerebral contact 11 is associated with a transmission track 12.

As can be seen in FIG. 2, in the example depicted the multilayer film 5 in the distal part 101 includes, in addition to the substrate 20, a first conductive layer 10a deposited directly on the substrate 20 and forming the transmission tracks 12. This first conductive layer 10a has a thickness Ec of 10 μm and an overthickness Ei of 25 μm at the ends 15 of the transmission tracks 12.

The multilayer film 5 further includes the insulative layer 30, the thickness of which is Ei, also deposited on the substrate 20, and covers the whole of substrate 20 not covered by the transmission tracks 12, except for the ends 15 of the transmission tracks 12.

The insulative layer 30 espouses the relief formed at the level of the transmission tracks 12.

In this example the or each insulative layer 30 includes a liquid crystal polymer material, for example the same material as the substrate 20.

As can be seen in FIG. 2, the multilayer film 5 includes a second conductive layer 10b of thickness Ey of 10 μm deposited on the insulative layer 30 and forming the intracerebral contacts 11.

Each transmission track 12 is in contact at its end 15 with an intracerebral contact 11. The intracerebral contacts 11 and the transmission tracks 12 are therefore connected by a via 31 in the insulative layer 30.

Note that, as described in more detail hereinafter, in the proximal part 103 of the probe 100 the multilayer film 5 includes the substrate 20, at least one conductive layer 10 forming the transmission tracks 12 and connector contacts 77 at the ends of the transmission tracks 12. The transmission tracks 12 are covered with at least one insulative layer 30. The connector contacts 77 enable electrical contact between the probe 100 and the external recording and/or stimulation and/or treatment device.

Finally, in the connecting part 102 of the probe 100 the multilayer film 5 includes the substrate 20, at least one conductive layer forming the transmission tracks 12 and at least one insulative layer 30 covering the transmission tracks 12.

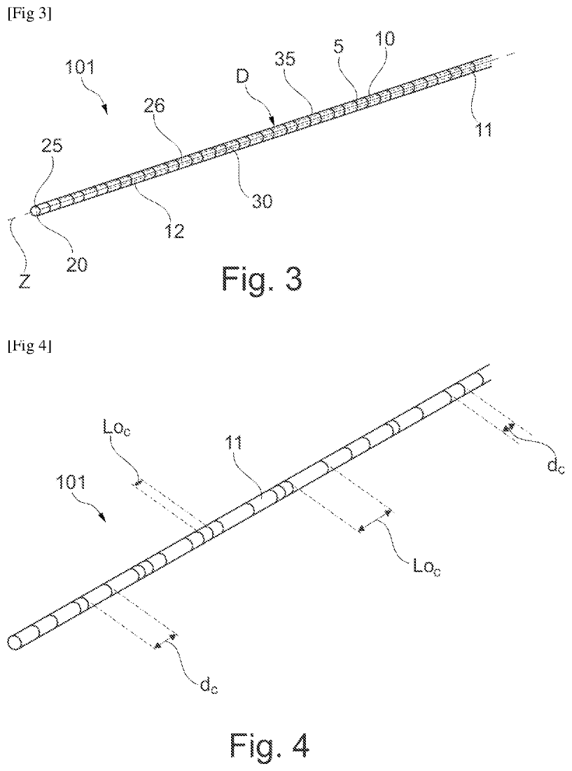

There is seen in FIG. 3 the cylinder formed by the distal part 101 defining an interior cavity 25 that has a circular section of outside diameter D of approximately 0.7 mm.

In this example, each intracerebral contact 11 extends over the whole of the circumference of the cylinder.

Moreover, in this example the distal part 101 includes a temperature sensor 35 formed by the conductive layer 10.

Various examples of the distal part 101 of a probe 100 have been depicted in FIGS. 4 to 7. To clarify the drawings the transmission tracks 12 have not been represented in these figures.

As can be seen in the FIG. 4 example the intracerebral contacts 11 have different lengths Lo_{c} and are spaced from one another by different distances d_{c}.

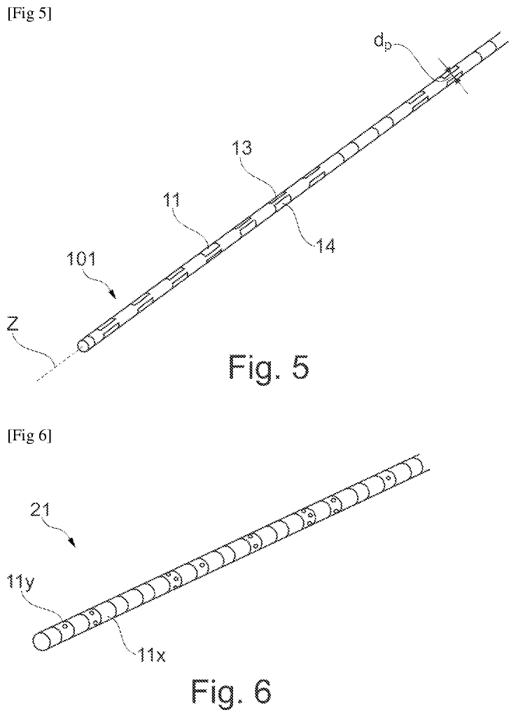

In the FIG. 5 example the intracerebral contacts 11 extend over only part of the circumference of the cylinder formed by the distal part 101. Each intracerebral contact 11 that extends transversely to the axis Z includes two parts 13 and 14 separated from one another by a space of width dₚ. In this example the spaces of width dₚ between the parts 13 and 14 of a plurality of intracerebral contacts 11 disposed one after the other are not aligned with one another along the axis Z, but are offset.

In the FIG. 6 example the distal part 101 includes intracerebral contacts 11x extending over the whole of the circumference of the cylinder formed by the distal part 101 and intracerebral contacts 11y of circular shape.

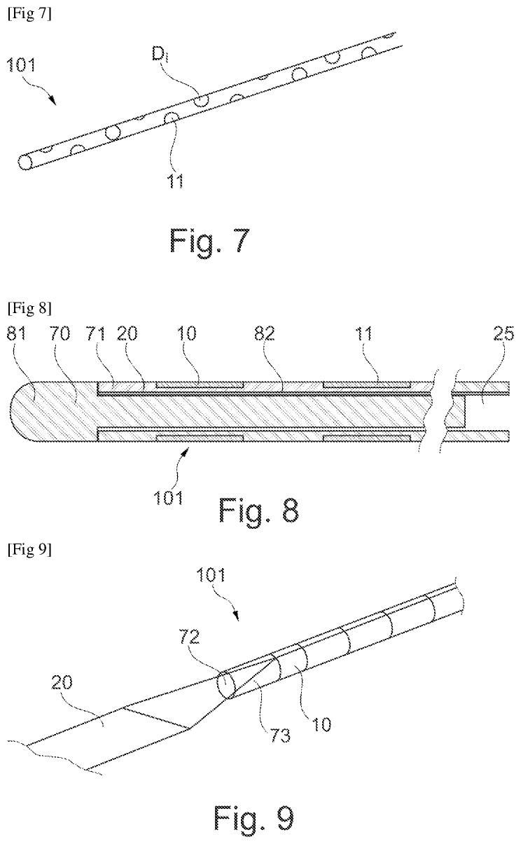

In the FIG. 7 example the distal part 101 includes intracerebral contacts 11 of circular shape with diameter Di equal to approximately 2 mm. The intracerebral contacts 11 are moreover disposed in a quincunx on the multilayer film 5.

Using intracerebral contacts 11 of circular shape makes it possible to produce probes 100 capable of producing a directional pulse or measurement.

In the embodiment depicted in FIG. 8 the probe 100 includes a distal stud 70, made of metal in this example, inserted in the interior cavity 25 from the distal end 71 and fixed by gluing or by welding or by mechanical fixing. The distal stud 70 then forms an intracerebral contact 81 independent of the other intracerebral contacts 11. In this example the distal stud 70 is in contact with a specific distal transmission track 82 deposited on the substrate 20.

Alternatively or additionally the interior cavity 25 may be partly filled with silicone charged with metal particles enabling an electrical connection to be made and/or established between the distal stud 70 and a transmission wire or a transmission track 12 in contact with the charged silicone.

In the FIG. 9 embodiment the distal part 101 of the probe 100 includes a plug 72 that has been inserted in the interior cavity 25 at the proximal end 73 of the distal part 101. This plug 72 is made of silicone, for example.

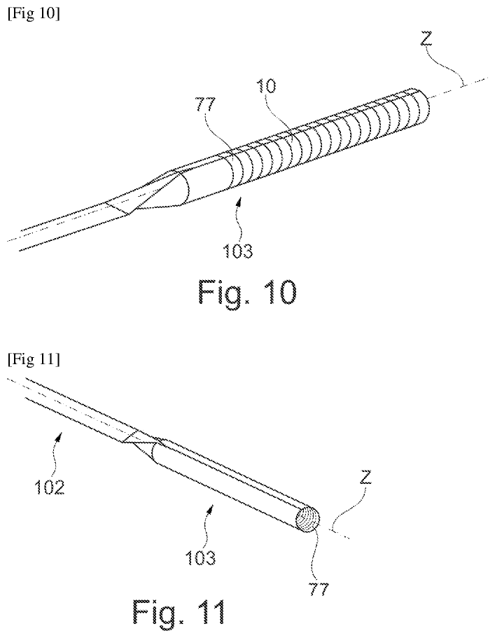

FIGS. 10 and 11 depict the proximal part 103 of the probe 100. The proximal part 103 of the probe 100 extends along the longitudinal axis Z and includes connector contacts 77 formed by the conductive layer 10, each connected to a single intracerebral contact 11 present on the distal part 101 by a single transmission track 12 on the proximal part 103, the connecting part 102 and the distal part 101.

In the FIG. 10 example the connector contacts 77 of the proximal part 103 are disposed on the exterior surface of the cylinder formed by the proximal part 103.

Alternatively, in the FIG. 11 example the connector contacts 77 of the proximal part 103 are disposed on the interior surface of the cylinder formed by the proximal part 103.

The connecting part 102 includes transmission tracks 12 extending the transmission tracks 12 of the distal part 101 and the proximal part 103. Each intracerebral contact 11 is connected to a single connector contact 77 by a single transmission track 12 extending part of the distal part 101, the connecting part 102 and part of the proximal part 103.

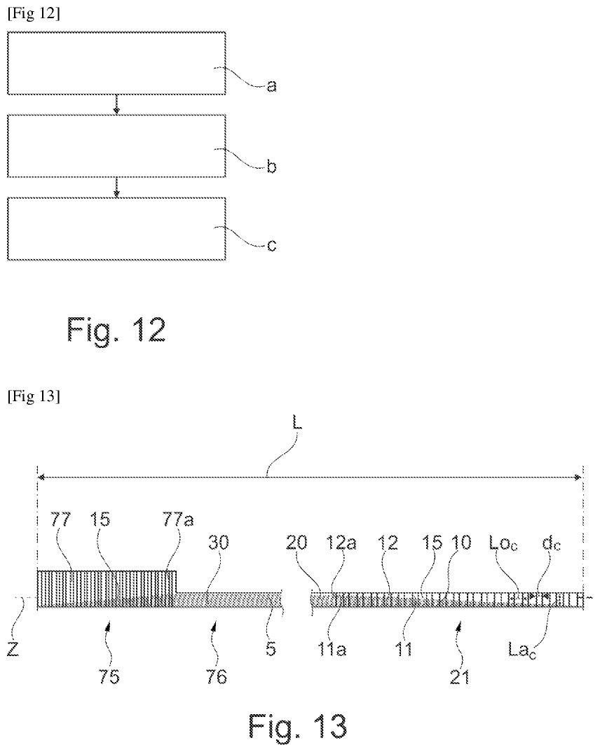

An example of a method of manufacturing a multi-contact probe 100 according to the invention for intracerebral functional investigation including three steps a, b and c has been depicted in FIG. 12.

As can be seen in FIGS. 2 and 13, in the first step a of producing the multilayer film 5 at least one conductive layer 10, in this example several of them made in this example of gold or with a coating of platinum and iridium, is/are deposited flat on a substrate 20 made from a liquid crystal polymer (LCP) material.

In this example the substrate 20 is cut out before step a and has a length L equal to approximately 1000 mm. As can be seen in FIG. 13 the substrate 20 is elongate, flat and extends along a longitudinal axis Z. The substrate 20 has a first part 21 intended to form the distal part 101 of the multi-contact probe 100 for intracerebral functional investigation, a part 76 intended to form the connecting part 102, and a second part 75 intended to form the proximal part 103 of the probe 100. As can be seen in FIG. 2, in this example the substrate 20 has a thickness Es transverse to the axis Z equal to 50 μm.

In this example the various conductive layers 10 are deposited in order to form a plurality of intracerebral contacts 11 in the first part 21, a plurality of connector contacts 77 in the second part 75, and a plurality of transmission tracks 12. In this example each intracerebral contact 11 is associated with a transmission track 12 and a connector contact 77.

In the example depicted the conductive layers 10 are deposited in the manner described with reference to FIG. 2. The conductive layers 10 and the insulative layers 30 are deposited by stacking them on the substrate 20 and then compressed and heated.

Each intracerebral contact 11 is connected to a single connector contact 77 by a single transmission track 12. For example, the intracerebral contact 11a is connected to the connector contact 77a by the transmission track 12a.

In the second step b of the method according to the invention a cylinder is formed extending along a longitudinal axis Z from a first part 21 of the multilayer film 5 intended to form said distal part 101 of the probe 100 in order to obtain the latter.

In the third step c of the method according to the invention a cylinder is formed extending along the longitudinal axis Z from a second part 75 of the multilayer film 5 and intended to form the proximal part 103 of the probe 100 in order to obtain the latter.

In the example depicted in FIG. 13 the intracerebral contacts 11 have a width La_{c} of 2.5 mm extending over the whole of the width of the substrate 20 transversely to the axis Z and a length Lo_{c} of 2 mm measured parallel to the axis Z. They are identical to one another. The transmission tracks 12 have a width of 50 μm. The intracerebral contacts 11 are regularly spaced from one another by a distance d_{c} equal to approximately 1.5 mm.

In this example the connector contacts 77 are deposited over a width greater than the length La_{c} of the intracerebral contacts 11. They are regularly spaced and have a constant length measured parallel to the axis Z.

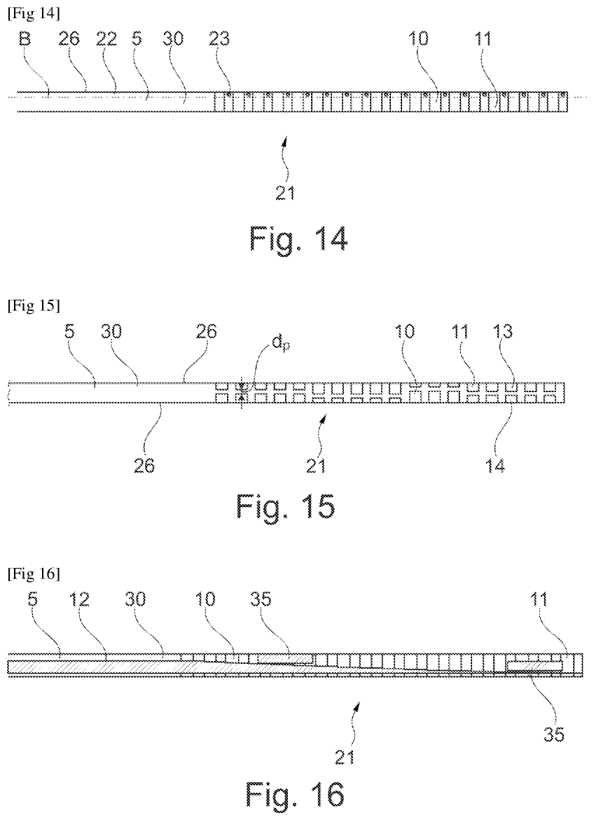

There have been depicted in FIGS. 14 to 16 various examples of the first part 21 of the multilayer film 5 intended to form the distal part 101 of a probe 100 after step a and before step b. To clarify the drawings the transmission tracks 12 have again not been represented in these figures.

In FIG. 14 the part 21 includes eighteen intracerebral contacts 11 with the same dimensions. The substrate 20 includes windows 23 passing through it on a lateral edge 22. The lateral edge 22 extends from a lateral end 26 of the substrate 20 as far as a part of the substrate 20 near this lateral end 26, in this example as far as the line B.

In this FIG. 14 example step a is performed in such a manner as to deposit the conductive layers 10 and the insulative layers 30 outside the windows 23. The multilayer film 5 then itself includes windows 23. The use of windows 23 can make it possible to facilitate fixing the lateral edges 22 together, as described hereinafter.

In the FIG. 15 example the intracerebral contacts 11 do not extend over the whole of the width of the substrate 20 transversely to the axis Z, but over only a part of the latter. Each intracerebral contact 11 that extends transversely to the axis Z includes two parts 13 and 14 separated from one another by a space of width dₚ. Each part 13 or 14 extends from a lateral end 26 of the substrate 20. The spaces of width dₚ between the parts 13 and 14 of a plurality of intracerebral contacts disposed one after the other are not aligned with one another along the axis Z in this example, but offset.

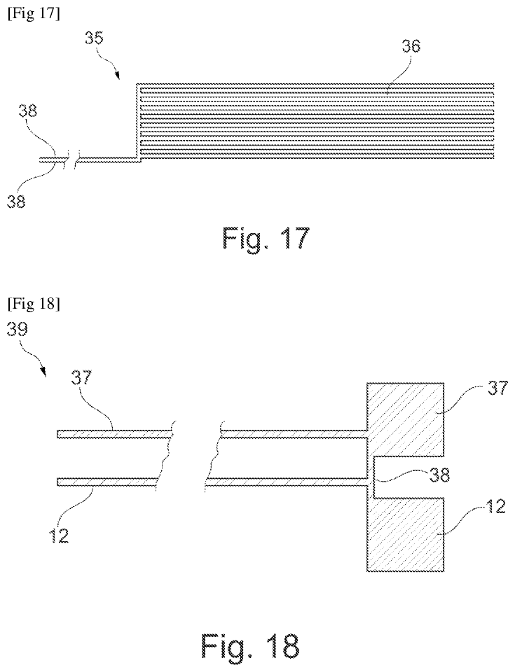

In the example depicted in FIG. 16 there have been formed on the first part 21 of the multilayer film 5, in addition to the intracerebral contacts 11 and the transmission tracks 12, two resistance type temperature sensors 35 shown to a larger scale in FIG. 17.

Each temperature sensor 35 includes a long track 36 of zig-zag shape connected to two tracks 37 and 38. As depicted in FIG. 18, the track 38 is connected to a transmission track 12 connected to an intracerebral contact 11.

The resistance of the circuit 39 between the track 37 and the transmission track 12 connected to the track 38 varies as a function of temperature.

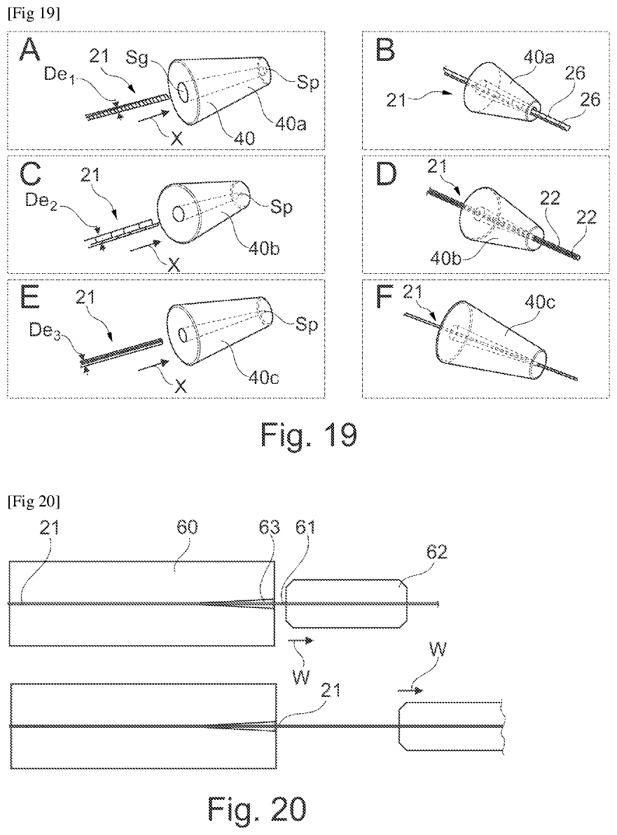

An example of performing step b has been depicted in FIG. 19. In this example step b consists in rolling the first part 21 with the aid of three truncated cones 40.

In this embodiment the aim is to move closer edge to edge the lateral ends 26 of the lateral edges 22.

The multilayer film 5 is flat before the multilayer film 5 is inserted in the first cone 40a (view A). The diameter De¹ of the equivalent circular section of the first part 21 of the multilayer film 5 is represented in dashed line. The multilayer film 5 is inserted in the truncated cone 40a in the direction represented by the arrow X through the largest section Sg as far as the smallest section Sp.

As can be seen in view B, during insertion the first part 21 of the multilayer film 5 is curved to form a circular arc. The diameter De₂ of the equivalent circular section formed after insertion in the truncated cone 40a is less than the diameter De¹ before insertion.

As can be seen in view C, the first part 21 of the multilayer film 5 is then withdrawn from the truncated cone 40a to be inserted in the direction X in the greater section Sg of a truncated cone 40b having a smaller section Sg less than the smaller section Sg of the truncated cone 40a.

As can be seen in view D, after insertion in the truncated cone 40b the first part 21 is rolled further on itself and the diameter De₃ of the equivalent circular section is less than the diameter De₂.

As can be seen in view E, the first part 21 of the multilayer film 5 is then withdrawn from the truncated cone 40b to be inserted in the direction X in the greater section Sg of a truncated 40c having a smaller section Sg less than the smaller section Sg of the truncated cone 40b.

As can be seen in view F, after insertion in the truncated cone 40c the first part 21 is rolled further on itself and the two lateral edges 22 come into contact.

The cylinder formed in this way after step b has a circular section, forms the interior cavity 25 and, in this example, has an outside diameter D of approximately 0.8 mm.

Each intracerebral contact 11 extends over the whole of the circumference of the cylinder formed in this example.

The first part 21 of the multilayer film 5 therefore forms the distal part 101 of the intracerebral functional investigation probe 100.

It is likewise possible to perform step c by rolling the second part 75 of the multilayer film 5 intended to form the proximal part 103 of the probe 100 with the aid of truncated cones in a manner analogous to what has just been described.

In order to maintain the cylindrical shape of the first part 21 of the multilayer film 5 the interior cavity 25 may be filled with silicone in this example. The filling process is depicted in FIG. 20 and described hereinafter.

The first part 21 of the multilayer film 5 is inserted in a cavity 63 opening onto one end of a mold 60. A mandrel 61 is inserted in the cavity formed by the first part 21 of the multilayer film 5. The mandrel 61 is connected to a store 62 of silicone that can also serve as holding means. As the silicone is injected the mandrel 61 is progressively withdrawn from the interior cavity 25 in the direction W.

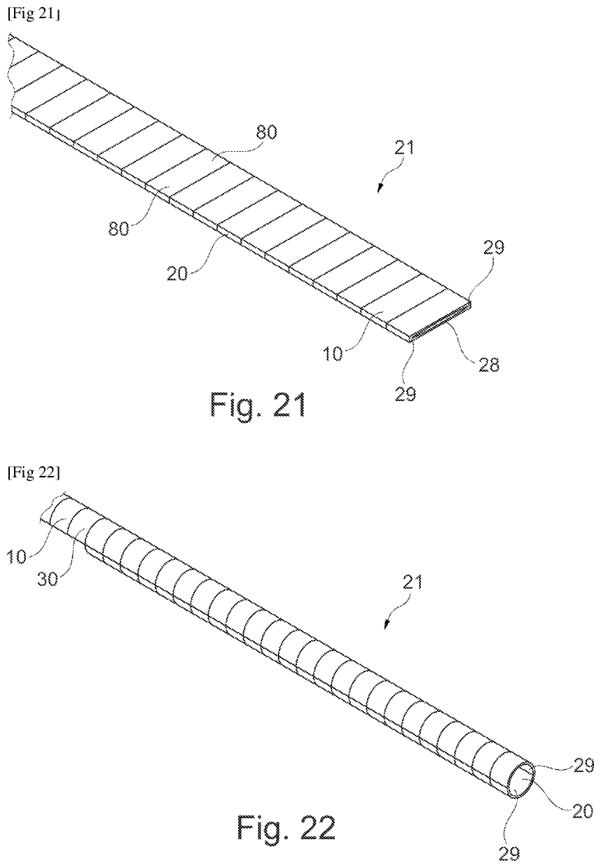

Other examples of the method according to the invention are described with reference to FIGS. 21 to 24.

In the embodiment depicted in FIGS. 21 and 22, in the first part 21 of the multilayer film 5 the substrate 20 has within its thickness an interior cavity 28 closed laterally but open at at least one longitudinal end, in the example depicted at both longitudinal ends.

In this case the substrate 20 may be manufactured before step a by superposing two layers 29 of liquid crystal polymer material and then welding their transverse edges 80.

In this example step b is performed by filling the interior cavity 28 of the substrate 20 with at least one biocompatible material in such a manner as to form the cylindrical distal part 101.

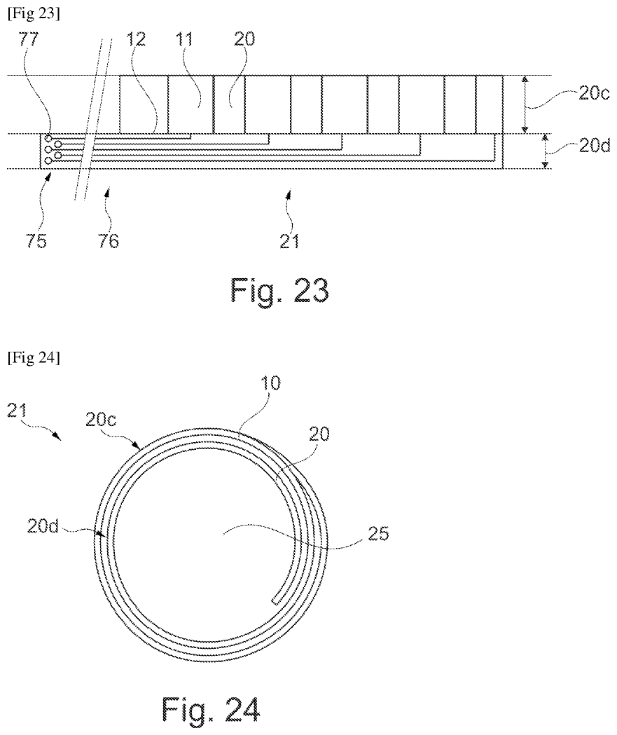

In the embodiment depicted in FIGS. 23 and 24 the intracerebral contacts 11 are deposited during step a in a part 20c of the substrate 20 and the transmission tracks 12 and the connector contacts 77 in a part 20d of the substrate 20, the parts 20c and 20d being laterally adjacent but separate. Each intracerebral contact 11 is connected to a single connector 77 by a single transmission track 12. The transmission tracks 12 do not cross over.

As can be seen in FIG. 24, in this example step b of the method is performed by rolling the multilayer film 5 on itself in such a manner that the part 20c forms the exterior periphery of the cylinder so formed. The part 20d is disposed inside the interior cavity 25 formed by the cylinder.

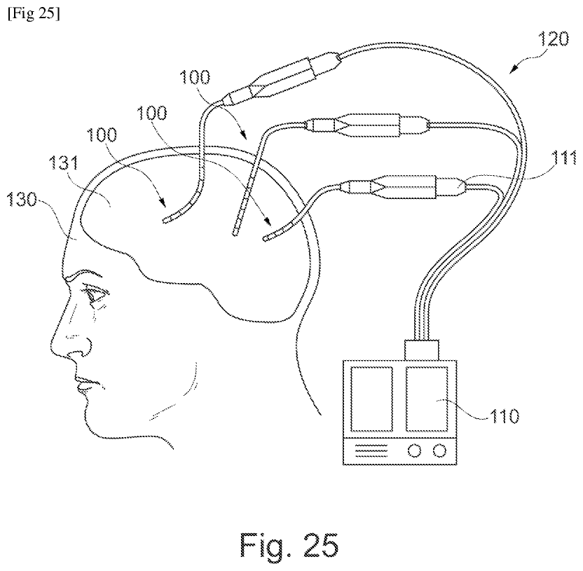

As depicted in FIG. 25, the probe 100 can be connected to a device 110 for recording and/or stimulation and/or treatment of the patient with the aid of a connector 111 to form a multi-contact device 120 for intracerebral functional investigation and/or stimulation and/or treatment, in particular by radiofrequencies.

The device 110 for recording and/or stimulation and/or treatment of the patient also enables processing of data received at the connector contacts 77 transmitted by the transmission tracks 12 from the intracerebral contacts 11.

As depicted in FIG. 25, a device 120 of this kind can make it possible to perform intracerebral functional investigation or treatment or stimulation including the following steps:

Step x: inserting at least one multi-contact intracerebral functional investigation probe 100 as described above, in this example three probes 100, in the brain 131 of a patient 130,

Step y: connecting the probes 100 to at least one recording and/or stimulation and/or treatment device 110, and

Step z: measuring cerebral electrical activity and/or performing electrical stimulation in the brain 131 of the patient 130 and/or performing electrical treatment in the brain 131 of the patient 130.

The invention is not limited to the examples that have just been described.

In particular, the method may include a step of finishing the conductive layer, for example an etching step.

The number of intracerebral contacts may be different, for example between 2 and 60 inclusive.

The conductive layer 10 may be deposited by some other process, in particular by a thin layer deposition process.

The multilayer film 5 may be rolled on itself in a different manner, for example using more or fewer truncated cones.

The distal stud 70 may be connected to a device for recording and/or stimulation and/or treatment of the patient by a connecting wire.

The various layers deposited on the substrate 20 may have different thicknesses.

It is possible to deposit on the substrate 20 a plurality of conductive and/or insulative layers, for example between 2 and 10 conductive and/or insulative layers.

The insulative layers 30 may include some other polymer material, in particular some other biocompatible polymer material, such as polyamide.

In a variant that is not depicted the rolling of the first part 21 of the multilayer film 5 is performed in such a manner as partly to superpose the lateral edges 22, for example with the aid of truncated cones.

In the situation where the rolling of the first part 21 of the multilayer film 5 is performed in such a manner as partly to superpose the lateral edges 22, the lateral edges 22 may be glued or welded together.

1-15. (canceled)

16. A multi-contact probe for intracerebral functional investigation and/or stimulation and/or treatment by radiofrequencies, comprising:
at least one distal part of cylindrical shape comprising at least one intracerebral contact intended to be implanted in the brain of a patient,
at least one proximal part of cylindrical shape comprising at least one connector contact intended to be connected to at least one device for recording and/or stimulation and/or treatment outside the body of the patient, and
at least one connecting part of non-cylindrical shape configured to connect the distal part and the proximal part,
wherein one or more of the at least one distal part, the at least one proximal part, and the at least one connecting part comprises a multilayer film comprising a substrate and at least one conductive layer deposited on the substrate,
wherein the substrate comprises at least one polymer material, the at least one conductive layer comprises at least one transmission track, at least one intracerebral contact in the distal part, at least one connector contact in the proximal part, wherein each of the at least one transmission track is connected to the at least one intracerebral contact in the distal part and the at least one connector contact in the proximal part.

17. The probe of claim 16, wherein the multilayer film comprises at least one insulative layer of a polymer material deposited on the substrate, wherein the at least one insulative layer at least partially covers the at least one transmission track.

18. The probe of claim 16, comprising intracerebral contacts in a total number between 1 and 60.

19. The probe of claim 16, wherein the substrate comprises at least one liquid crystal polymer (LCP) material.

20. The probe of claim 16, wherein an interior cavity formed by a cylinder of the distal part is at least partially filled with at least one of a glue, a polymer material, or a composite material.

21. The probe of claim 16, wherein the distal part comprises at least one temperature sensor.

22. The probe of claim 16, wherein the distal part comprises a distal end and a distal stud configured to close the distal end.

23. The probe of claim 16, wherein the distal part comprises a proximal end and a plug configured to close the proximal end.

24. The probe of claim 16, wherein the distal part comprises the multilayer film.

25. A method of manufacturing a multi-contact probe for intracerebral functional investigation and/or stimulation and/or treatment by radiofrequencies, comprising:
a) forming a multilayer film by depositing flat, on at least a part of a substrate, at least one conductive layer to form at least one intracerebral contact, at least one connector contact, and at least one transmission track, wherein each of the at least one transmission track is connected to one of the at least one intracerebral contact and one of the at least one of a connector contact;
b) forming a cylinder that extends along a longitudinal axis from at least a first part of the multilayer film to form a distal part of a probe configured to be implanted in the brain of a patient; and
c) forming a cylinder that extends along the longitudinal axis from at least a second part of the multilayer film to form a proximal part of the probe configured to be connected to at least one device for recording and/or stimulation and/or treatment outside the patient.

26. The method of claim 25, further comprising, after depositing the at least one conductive layer on the substrate, compressing and/or heating the at least one conductive layer on the substrate to fix the at least one conductive layer on the substrate.

27. The method of claim 25, wherein the first part and/or the second part of the multilayer film comprises at least one lateral edge, and the method further comprises at least partially rolling the first part and/or said second part of the multilayer film to at least partially superpose the respective lateral edge.

28. The method of claim 25, wherein the first part and/or the second part of the multilayer film comprises at least one lateral edge, and the method further comprises at least partial rolling the first part and/or the second part of the multilayer film to bring the lateral edge of the first part and the lateral edge of the second part into edge-to-edge contact.

29. The method of claim 25, wherein the substrate comprises at least one window on at least one of the lateral edge of the first part of the multilayer film and the lateral edge of the second part of the multilayer film, step a being performed in such a manner as to deposit the at least one conductive layer outside the at least one window.

30. The method of claim 29, wherein the first part and/or the second part of the multilayer film is rolled by successively inserting the respective part of the multilayer film into at least one truncated cone, such that the diameter of one equivalent circular section of the respective part of the multilayer film is reduced after successive insertion in each respective truncated cone.

31. The method of claim 25, wherein the substrate has an interior cavity closed laterally but open at least one longitudinal end within its thickness, and the method further comprises filling the interior cavity of the substrate with at least one material.

32. A multi-contact device for intracerebral functional investigation and/or stimulation and/or treatment by radiofrequencies, comprising at least one multi-contact probe for intracerebral functional investigation, and at least one device for recording and/or stimulation and/or treatment of the patient connected to the at least one probe,
wherein the at least one probe comprises:
at least one distal part of cylindrical shape comprising at least one intracerebral contact intended to be implanted in the brain of a patient,
at least one proximal part of cylindrical shape comprising at least one connector contact intended to be connected to at least one device for recording and/or stimulation and/or treatment outside the body of the patient, and
at least one connecting part of non-cylindrical shape configured to connect the distal part and the proximal part,
wherein the at least one connecting part comprises a multilayer film comprising a substrate and at least one conductive layer deposited on the substrate,
wherein the substrate comprises at least one polymer material, the at least one conductive layer comprises at least one transmission track, at least one intracerebral contact in the distal part, and at least one connector contact in the proximal part,
wherein each of the at least one transmission track is connected to the at least one intracerebral contact in the distal part and the at least one connector contact in the proximal part.
